(19) United States
(12) Patent Application Publication
(10) Pub. No.: US 2021/0253822 A1
(43) Pub. Date: Aug. 19, 2021

(54) BARRIER LAYER AND GAS SENSOR INCLUDING THE BARRIER LAYER

(71) Applicant: Nuvoton Technology Corporation, Hsinchu Science Park (TW)

(72) Inventor: Ming-Chih TSAI, Taichung City (TW)

(21) Appl. No.: 17/164,055

(22) Filed: Feb. 1, 2021

(30) Foreign Application Priority Data
Feb. 18, 2020 (TW) 109105088

(51) Int. Cl. C08J 9/28; G01N 27/22; G01N 33/00

(52) U.S. Cl. CPC C08J 9/286; G01N 27/223; C08J 2300/104; C08J 2300/102; G01N 33/0027

(57) ABSTRACT

A barrier layer is provided. The barrier layer includes a porous structure, which includes a polymer material, an oxide and a fluoro-containing material. A chemical bond is formed between the oxide and the polymer material. The fluoro-containing material, the polymer material and the oxide are assembled as a composite structure.

### CROSS REFERENCE TO RELATED APPLICATIONS

This application claims priority of Taiwan Patent Application No. 109105088, filed Feb. 18, 2020, the entirety of which is incorporated by reference herein.

### BACKGROUND

### Field of the Invention

The present invention relates to a barrier layer and a gas sensor including the barrier layer, and in particular to a barrier layer having polymer, oxide and fluoro-containing material, and a gas sensor including the barrier layer.

### Description of the Related Art

Currently, environment sensors are commonly used in electronic devices for detecting pressure, humidity, or various gases. These sensors need to be packaged in special customized material, such that the sensors may be exposed to the environment for performing detection, preventing failure due to liquid, water vapor, or dust in the environment. Generally, an air-permeable membrane may serve as a protective structure for the sensors.

However, the cost of the sensors with a waterproofing and/or dust-proofing function is quite high. Therefore, reducing the cost and popularizing waterproof and/or dust-proof sensors is still an important topic.

### BRIEF SUMMARY

Some embodiments of the present disclosure provide a barrier layer including: a porous structure, which includes a polymer material, an oxide and a fluoro-containing material. A chemical bond is formed between the oxide and the polymer material. The fluoro-containing material, the polymer material and the oxide are assembled as a composite structure.

Some embodiments of the present disclosure provide a gas sensor, including: the aforementioned barrier layer.

A detailed description is given in the following embodiments with reference to the accompanying drawings.

### BRIEF DESCRIPTION OF THE DRAWINGS

The present disclosure can be more fully understood by reading the subsequent detailed description and examples with references made to the accompanying drawings, wherein:

FIG. 1A to 1E are perspective views illustrating a manufacturing process of a porous structure in accordance with some embodiments of the present disclosure.

FIG. 2 is an enlarged view illustrating the porous structure in accordance with some embodiments of the present disclosure.

FIG. 3 is an enlarged view illustrating the porous structure in accordance with some embodiments of the present disclosure.

FIG. 4 is an enlarged view illustrating the porous structure in accordance with some embodiments of the present disclosure.

FIG. 5 is an enlarged view illustrating a membrane in accordance with a comparative example of the present disclosure.

FIG. 6 is a diagram illustrating the relationship between relative humidity and capacitance of the porous structure in accordance with some embodiments of the present disclosure.

FIG. 7A to 7C are perspective views illustrating a manufacturing process of a gas sensor in accordance with some embodiments of the present disclosure.

### DETAILED DESCRIPTION OF THE INVENTION

The barrier layers and gas sensors of some embodiments of the present disclosure are described in the following description. The specific embodiments disclosed are provided to implement different types of some embodiments of the present disclosure. Specific elements and arrangements discussed in the following paragraphs are merely provided for simply and clearly describe some embodiments of the present disclosure. Of course, these elements and arrangements merely serve as examples without limiting the scope of the present disclosure. Repeating numerals or marks may be used in different embodiments. These repetitions are just for simply and clearly describe some embodiments of the present disclosure, but not imply any relationship between different embodiments and/or structures discussed below.

In the present specification, the terms “about,” “approximately,” or “substantially” are generally interpreted as within 20% of a given value or range, or as interpreted as within 10%, 5%, 3%, 2%, 1%, or 0.5% of a given value or range. The given value is an approximate value. That is, even if the terms “about,” “approximately,” or “substantially” are not recited in the description, it should be read as the same meaning as these terms are recited. In addition, the phrase “in a range from a first value to a second value” means that the range includes the first value, the second value and other values between the former two.

Unless defined otherwise, all teams (including technical and scientific terms) used herein have the same meaning as commonly understood by one of ordinary skill in the art to which this disclosure belongs. It should be appreciated that, in each case, the term, which is defined in a commonly used dictionary, should be interpreted as having a meaning that conforms to the relative skills of the present disclosure and the background or the context of the present disclosure, and should not be interpreted in all idealized or overly formal manner unless so defined in the present disclosure.

According to some embodiments of the present disclosure, a porous structure is provided and includes a polymer material, an oxide and a fluoro-containing material. A chemical bond is formed between the oxide and the polymer material. The fluoro-containing material, the polymer material and the oxide are assembled as a composite structure.

In some embodiments, the polymer material is a macromolecular material including a hydroxyl group (—OH), and for example, includes repeating units as follows:

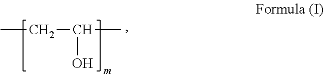

In some embodiments, m is an integer a range from 1 to 10000, but the present disclosure is not limited thereto. In some embodiments, the polymer material comprises repeating units as follows:

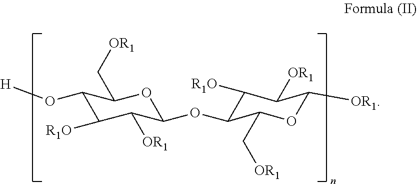

In some embodiments, n is an integer in a range from 1 to 10000, R₁ is (CH₂)ᵢH, (OC₂H₄)ⱼH, (OC₃H₆)ₖH, or a combination thereof, i is an integer in a range from 0 to 24, j is an integer in a range from 0 to 18, and k is an integer in a range from 0 to 12. However, the present disclosure is not limited thereto. It should be noted that the plurality of R₁ are the same as or different from each other, or partially the same but partially different.

For example, the oxide may be graphene oxide, reduced graphene oxide, silicon oxide, metal oxide, or metal bronze compound including precursors of the aforementioned metal oxide. In some embodiments, the oxide includes a unit as follows: AxMyOz Formula (III).

In some embodiments, A includes at least one cation, M includes at least one cation of transition metal and metalloid, or carbon ions. Y is the sum of the numbers of the at least one of transition metal ions, metalloid ions, and carbon ions. Z is the number of the oxygen ion. The values of x, y and z may equalize charge number of the Formula (III).

In some embodiments, A includes at least one cation, such as hydrogen ion, alkali metal ion, alkaline earth metal ion, rare earth metal ion, ammonium ion, or a combination thereof. For example, the cation may be hydrogen (H) ion, lithium (Li) ion, sodium (Na) ion, potassium (K) ion, rubidium (Rb) ion, cesium (Cs) ion, silver (Ag) ion, or a combination thereof. However, in the present disclosure, the cation that serves as A is not limited to the cations listed above. M includes at least one ion of a transition metal and a metalloid, or a carbon ion. The transition metal may be tin (Sn), titanium (Ti), zirconium (Zr), cerium (Ce), hafnium (Hf) molybdenum (Mo), tungsten (W), vanadium (V), copper (Cu), iron (Fe), Cobalt (Co), nickel (Ni), manganese (Mn), niobium (Nb), tantalum (Ta), rhenium (Re), ruthenium (Ru), platinum (Pt), or a combination thereof, but the present disclosure is not limited thereto. The metalloid may be silicon (Si), boron (B), germanium (Ge), arsenic (As), or a combination thereof, but the present disclosure is not limited thereto. M may also be carbon (C), but the present disclosure is not limited thereto.

In some embodiments, the fluoro-containing material may be a sulfonated perfluorinated compounds (PFCs), a sulfonated fluoropolymer, or a phosphated perfluoroalkane compound. For example, the above fluorine-containing material may include, for example, a C4-C18 perfluoroalkyl chain formed by a fluorocarbon with a carbon number between 4 and 18, and a polytetrafluoroethylene (PTFE) formed by a fluorocarbon, and functional groups derived from, for example, sulfonic acid and phosphoric acid.

In order to make the above and other objects, features, and advantages of the present disclosure more obvious and understandable, a few examples are described below in detail as follows, but they are not intended to limit the scope of the present disclosure.

Embodiment 1: Fabrication of AxMyOz-C—F Composite Structure Membrane

First, the aforementioned polymer material is prepared as a solution with a concentration of 0.001%-20%. In some embodiments, the aforementioned polymer material can be prepared as a solution of about 0.3125%, about 0.625%, about 1.25%, about 2.5%, about 5%, or about 10%. For example, the solution may be a 0.1%-15% polyvinyl alcohol solution, a 0.1%-15% methyl cellulose solution, a 0.1%-15% sodium carboxymethyl cellulose solution, a 0.1%-15% hydroxyethyl cellulose solution, a 0.1%-15% hydroxyethyl methyl cellulose solution, a 0.1%-15% hydroxypropyl cellulose solution, a 0.1%-15% hydroxypropyl methylcellulose solution, a 0.1%-15% nanocellulose solution, or a 0.1%-15% aqueous solution that is a combination thereof, but it is not limited thereto. Then, an active metal bronze compound AxMyOz is grafted on the surface of the polymer material, and an oxide-polymer composite AxMyOz-C is formed after dehydration, condensation, and/or other steps are performed, but it is not limited thereto.

In the present embodiment, the metal bronze-based compound is grafted on the hydroxyl group (—OH) of the polymer material. Next, a 0.01%-10% fluoro-containing material including sulfonated fluoropolymer (such as perfluorosulfonic acid (PFSA)/polytetrafluoroethylene (PTFE) copolymer) is used to be assembled with the oxide-polymer complex AxMyOz-C to form a composite structure AxMyOz-C—F.

In the present embodiments, assembly of the composite structure AxMyOz-C—F is adjusted by using a highly volatile solvent system. Highly volatile substances (such as toluene, xylene, methyl ethyl ketone, acetone, propylene glycol methyl ether, propylene glycol methyl ether acetate, water, methanol, alcohol, isopropyl alcohol, or a combination thereof) serve as highly volatile solvents. The aforementioned composite AxMyOz-C—F is coated or deposited on a substrate via this highly volatile solvent system, the solvent is evaporated under a well-controlled environment, and then an annealing treatment is performed to obtain a dark brown membrane. The annealing treatment is, for example, performed in the atmosphere or in a nitrogen atmosphere at a temperature from 25° C. to 300° C. for 5 minutes to 12 hours. For example, in some embodiments, the annealing treatment is performed at 80° C. for 12 hours. In some embodiments, the annealing treatment is performed at 100° C. for 3 hours. In some embodiments, the annealing treatment is performed at 120° C. for 90 minutes. In some embodiments, the annealing treatment is performed at 150° C. for 60 minutes. In some embodiments, the annealing treatment is performed at 180° C. for 30 minutes. However, the present disclosure is not limited thereto. It should be realized that the above or other suitable annealing treatment may be adopted in the embodiments of the present disclosure as required, and it will not be described in detail below.

Embodiment 2: Fabrication of AxMyOz-C—F Composite Structure Membrane

First, the aforementioned polymer material is prepared as a solution with a concentration of 0.001%-20%. For example, the solution may be a 0.1%-15% polyvinyl alcohol solution, a 0.1%-15% methyl cellulose solution, a 0.1%-15% sodium carboxymethyl cellulose solution, a 0.1%-15% hydroxyethyl cellulose solution, a 0.1%-15% hydroxyethyl methyl cellulose solution, a 0.1%-15% hydroxypropyl cellulose solution, a 0.1%-15% hydroxypropyl methylcellulose solution, a 0.1%-15% nanocellulose solution, or a 0.1%-15% aqueous solution that is a combination thereof, but it is not limited thereto. Then, an active metal bronze compound AxMyOz is grafted on the surface of the polymer material, and an oxide-polymer composite AxMyOz-C is formed after dehydration, condensation, and/or other steps are performed.

In the present embodiment, the metal bronze-based compound is grafted on the hydroxyl group (—OH) of the polymer material. Next, a 0.01%-10% fluoro-containing material including phosphorylated perfluoroalkane compounds (e.g. alkyl phosphate ester fluorosurfactant) is used to be assembled with the oxide-polymer complex AxMyOz-C to form a composite structure AxMyOz-C—F.

In the present embodiments, assembly of the composite structure AxMyOz-C—F is adjusted by using a highly volatile solvent system. Highly volatile substances (such as toluene, xylene, methyl ethyl ketone, acetone, propylene glycol methyl ether, propylene glycol methyl ether acetate, water, methanol, alcohol, isopropyl alcohol, or a combination thereof) serve as highly volatile solvents. The aforementioned composite AxMyOz-C—F is coated or deposited on a substrate via this highly volatile solvent system, the solvent is evaporated under a well-controlled environment, and then an annealing treatment is performed to obtain a dark brown membrane. The annealing treatment is, for example, performed in the atmosphere or in a nitrogen atmosphere at a temperature from 25° C. to 300° C. for 5 minutes to 12 hours.

Embodiment 3: Fabrication of AxMyOz-C—F Composite Structure Membrane

First, the aforementioned polymer material is prepared as a solution with a concentration of 0.001%-20%. For example, the solution may be a 0.1%-15% polyvinyl alcohol solution, a 0.1%-15% methyl cellulose solution, a 0.1%-15% sodium carboxymethyl cellulose solution, a 0.1%-15% hydroxyethyl cellulose solution, a 0.1%-15% hydroxyethyl methyl cellulose solution, a 0.1%-15% hydroxypropyl cellulose solution, a 0.1%-15% hydroxypropyl methylcellulose solution, a 0.1%-15% nanocellulose solution, or a 0.1%-15% aqueous solution that is a combination thereof, but it is not limited thereto. Then, an active metal bronze compound AxMyOz is grafted on the surface of the polymer material, and an oxide-polymer composite AxMyOz-C is formed after dehydration, condensation, and/or other steps are performed.

In the present embodiment, the metal bronze-based compound is grafted on the hydroxyl group (—OH) of the polymer material. Next, one or more 0.01%-10% fluoro-containing material including sulfonated perfluoroalkane compounds (e.g. alkyl sulfonic acid/sulfonate fluorosurfactant), sulfonated fluoropolymers (e.g. perfluorosulfonic acid (PFSA)/polytetrafluoroethylene (PTFE) copolymer), or phosphorylated perfluoroalkane compounds (such as alkyl phosphate ester fluorosurfactant) may be assembled with the oxide-polymer composite AxMyOz-C to form a composite structure AxMyOz-C—F.

In the present embodiments, assembly of the composite structure AxMyOz-C—F is adjusted by using a highly volatile solvent system. Highly volatile substances (such as toluene, xylene, methyl ethyl ketone, acetone, propylene glycol methyl ether, propylene glycol methyl ether acetate, water, methanol, alcohol, isopropyl alcohol, or a combination thereof) serve as highly volatile solvents. The aforementioned composite AxMyOz-C—F is coated or deposited on a substrate via this highly volatile solvent system, the solvent is evaporated under a well-controlled environment, and then an annealing treatment is performed to obtain a dark brown membrane. The annealing treatment is, for example, performed in the atmosphere or in a nitrogen atmosphere at a temperature from 25° C. to 300° C. for 5 minutes to 12 hours.

### COMPARATIVE EXAMPLE 1

The aforementioned polymer material is prepared as a solution with a concentration of 0.001%-20%. For example, the solution may be a 0.1%-15% polyvinyl alcohol solution, a 0.1%-15% methyl cellulose solution, a 0.1%-15% sodium carboxymethyl cellulose solution, a 0.1%-15% hydroxyethyl cellulose solution, a 0.1%-15% hydroxyethyl methyl cellulose solution, a 0.1%-15% hydroxypropyl cellulose solution, a 0.1%-15% hydroxypropyl methylcellulose solution, a 0.1%-15% nanocellulose solution, or a 0.1%-15% aqueous solution that is a combination thereof, but it is not limited thereto. Then, an active metal bronze compound AxMyOz is grafted on the surface of the polymer material, and an oxide-polymer composite AxMyOz-C is formed after dehydration, condensation, and/or other steps are performed. In the present comparative example, the metal bronze-based compound is grafted on the hydroxyl group (—OH) of the polymer material.

In the present comparative example, assembly of the oxide-polymer composite AxMyOz-C is adjusted by using a highly volatile solvent system. Highly volatile substances (such as toluene, xylene, methyl ethyl ketone, acetone, propylene glycol methyl ether, propylene glycol methyl ether acetate, water, methanol, alcohol, isopropyl alcohol, or a combination thereof) serve as highly volatile solvents. The aforementioned composite AxMyOz-C is coated or deposited on a substrate via this highly volatile solvent system, the solvent is evaporated under a well-controlled environment, and then an annealing treatment is performed to obtain a dark brown membrane. The annealing treatment is, for example, performed in the atmosphere or in a nitrogen atmosphere at a temperature from 25° C. to 300° C. for 5 minutes to 12 hours.

### COMPARATIVE EXAMPLE 2

A commercially available (perfluoroalkyl)ethyl triethoxysilane (for example: (perfluorobutyl)ethyl triethoxysilane), (perfluorohexyl)ethyl triethoxysilane, (perfluorooctyl)ethyl triethoxysilane, a mixture including one or more thereof, but it is not limited thereto) is prepared as a solution with a concentration of 0.001%-20%, wherein highly volatile substances (e.g. perfluorohexane, hydrofluoroethers, toluene, xylene, methyl ethyl ketone, acetone, propylene glycol methyl ether, propylene glycol methyl ether acetate, water, methanol, alcohol, isopropanol, or a combination thereof) serve as a highly volatile solvent. Next, the target substrate is cleaned or surface-treated. The target substrate is immersed in the (perfluoroalkyl)ethyl triethoxysilane solution for 60 seconds to 60 minutes. Then, the target substrate is taken out and placed in a well-ventilated place at room temperature for 2 hours to 12 hours, or volatilize the solvent in a well-controlled environment for drying. After the aforementioned drying step, the above highly volatile substances is used to clean, and finally the finished product is taken out and dried in a well-ventilated environment at the room temperature.

TABLE 1
The comparison of hydrophilic or hydrophobic
contact angles measured in Embodiments 1-3

| | | | | Compar- | Compar- |
|---|---|---|---|---|---|
| | Embodi- | Embodi- | Embodi- | ative | ative |
| | ment 1 | ment 2 | ment 3 | example 1 | example 2 |
| The ratio | 0.05-5% | 0.05-5% | 0.05-5% | 0% | 100% |
| of fluoro- | | | | | |
| containing | | | | | |
| material | | | | | |
| Contact | 86 | 103 | 48 | None | 111 |
| angles | | | | (dis- | |
| (degrees) | | | | solved) | |
| Hydrophilic | partial | hydro- | hydro- | hydro- | hydro- |
| or | hydro- | phobic | philic | philic | phobic |
| hydrophobic | phobic | | | | |
| Waterproof | passable | great | bad | None | great |
| effect | | | | (dis- | |
| | | | | solved) | |

As shown in Table 1, compared to the comparative examples of the present disclosure, hydrophobic effect may be achieved without high ratio of fluoro-containing material in the membrane structures in the embodiments of the present disclosure. In addition, the contact angle and the porosity of the membrane structures in the embodiments of the present disclosure may be arbitrarily adjusted as required, such that the hydrophilic or hydrophobic degree of the membrane structures may be adjusted.

Embodiment 4: Fabrication of AxMyOz-C—F Composite Structure Membrane with Low Degree of Porosity in Microns

First, the aforementioned polymer material is prepared as a solution with a concentration of 0.001%-20%. For example, the solution may be a 0.1%-15% polyvinyl alcohol solution, a 0.1%-15% methyl cellulose solution, a 0.1%-15% sodium carboxymethyl cellulose solution, a 0.1%-15% hydroxyethyl cellulose solution, a 0.1%-15% hydroxyethyl methyl cellulose solution, a 0.1%-15% hydroxypropyl cellulose solution, a 0.1%-15% hydroxypropyl methylcellulose solution, a 0.1%-15% nanocellulose solution, or a 0.1%-15% aqueous solution that is a combination thereof, but it is not limited thereto. Then, an active metal bronze compound AxMyOz is grafted on the surface of the polymer material, and an oxide-polymer composite AxMyOz-C is formed after dehydration, condensation, and/or other steps are performed.

In the present embodiment, the metal bronze-based compound is grafted on the hydroxyl group (—OH) of the polymer material. Next, one or more of fluoro-containing material including sulfonated perfluoroalkane compounds (e.g. alkyl sulfonic acid/sulfonate fluorosurfactant), sulfonated fluoropolymers (e.g. perfluorosulfonic acid (PFSA)/polytetrafluoroethylene (PTFE) copolymer), or phosphorylated perfluoroalkane compounds (such as alkyl phosphate ester fluorosurfactant) may be assembled with the oxide-polymer composite AxMyOz-C to form a composite structure AxMyOz-C—F.

In the present embodiments, the composite structure AxMyOz-C—F may be formed by co-solvent controlled self-assembly to control the porosity. Highly volatile substances (such as toluene, xylene, methyl ethyl ketone, acetone, propylene glycol methyl ether, propylene glycol methyl ether acetate, water, methanol, alcohol, isopropyl alcohol, or a combination thereof) serve as a first solvent, lowly volatile substances (such as ethylene glycol, diethylene glycol ether, diethylene glycol butyl ether, triethylene glycol, propylene glycol, glycerol, isophorone, N-methylpyrrolidone, dimethyl sulfoxide (DMSO), or a combination thereof) serve as a second solvent, and the first solvent and the second solvent are combined as a composite solvent system. The aforementioned composite AxMyOz-C—F is coated or deposited on a substrate via the composite solvent system. The assembly is performed under a well-controlled environment by using the composite solvent polar system, wherein the ratio of the lowly volatile substances to the highly volatile substances is 2:100.

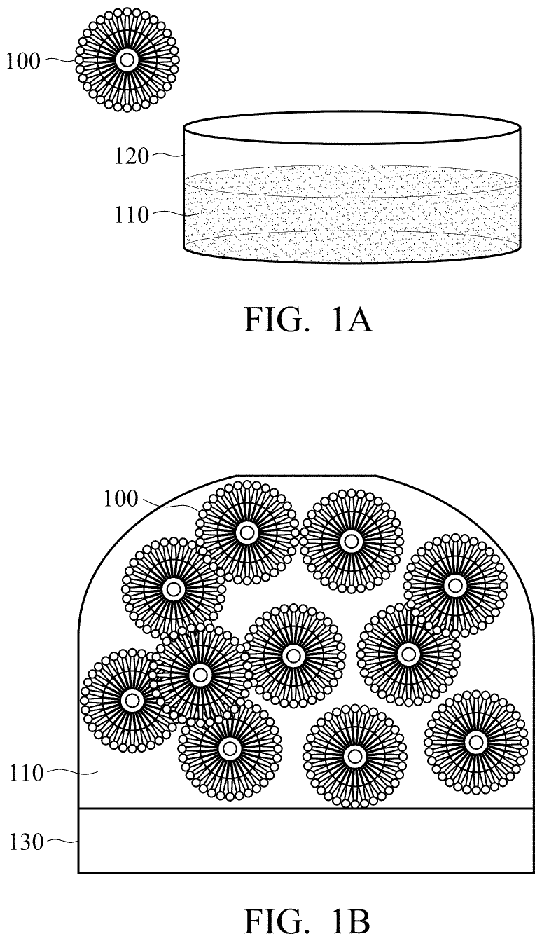

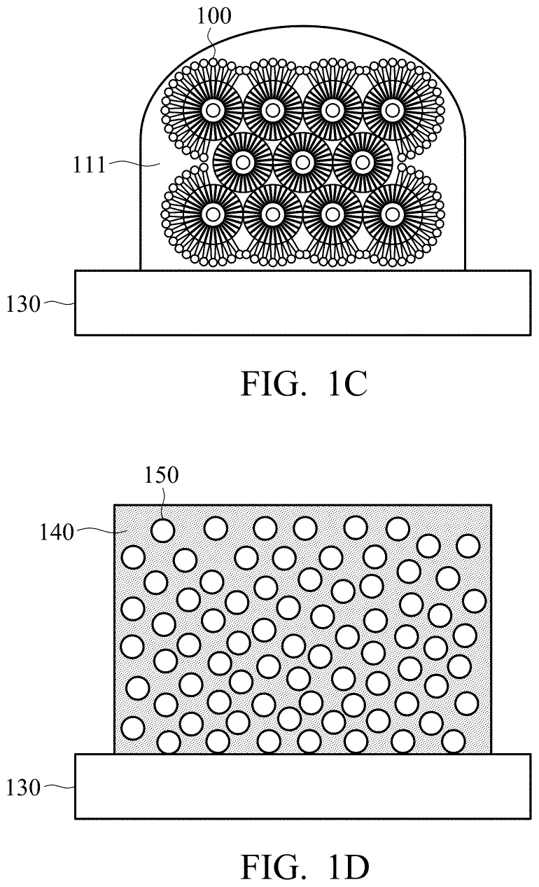

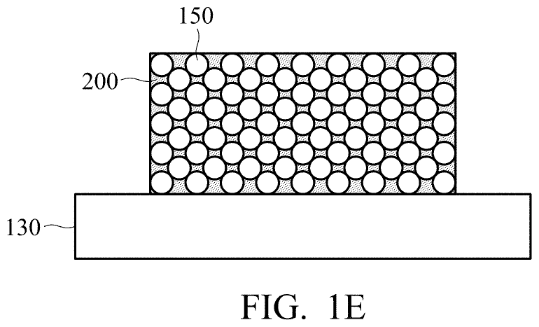

In some embodiments, as shown in FIG. 1A, a composite 100 (such as the composite AxMyOz-C—F) is added into a composite solvent system 110 in a container 120. The composite solvent system 110 may include any of the first solvent and the second solvent or any other suitable solvent. As shown in FIG. 1B, the composite solvent system 110 containing the composite 100 is coated on a substrate 130. Then, as shown in FIG. 1C, the more volatile first solvent may be volatilized faster than the less volatile second solvent, and the remaining second solvent forms a composite solvent system 111. The composite 100 may be assembled following its polarity. As shown in FIG. 1D, after the second solvent volatilizes, an assembly structure 140 having holes 150 is formed on the substrate 130.

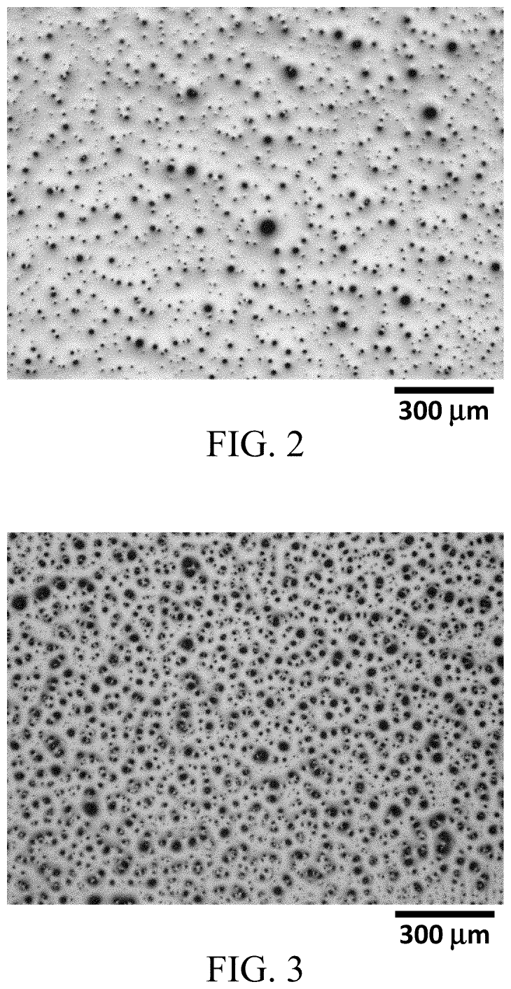

A porous structure may be obtained by adjusting the first phase highly volatile assembly, the second phase lowly volatile assembly, and the third phase annealing treatment. For example, as shown in FIG. 1E, a porous structure 200 having holes 150 is formed on the substrate 130. The annealing treatment is performed in the atmosphere or in a nitrogen atmosphere at a temperature from 25° C. to 300° C. for 5 minutes to 12 hours. The average diameter of the holes may be 11.94 μm, and the porosity thereof may be 8.96%. Please refer to FIG. 2, which is an enlarged view illustrating the porous structure in accordance with the present embodiment (Embodiment 4) of the present disclosure. It should be understood that the porosity discussed herein is a ratio of the area of the holes to the whole area of the membrane structure when observed by technical staff.

Embodiment 5: Fabrication of AxMyOz-C—F Composite Structure Membrane with Medium Degree of Porosity in Microns

The aforementioned polymer material is prepared as a solution with a concentration of 0.001%-20%. For example, the solution may be a 0.1%-15% polyvinyl alcohol solution, a 0.1%-15% methyl cellulose solution, a 0.1%-15% sodium carboxymethyl cellulose solution, a 0.1%-15% hydroxyethyl cellulose solution, a 0.1%-15% hydroxyethyl methyl cellulose solution, a 0.1%-15% hydroxypropyl cellulose solution, a 0.1%-15% hydroxypropyl methylcellulose solution, a 0.1%-15% nanocellulose solution, or a 0.1%-15% aqueous solution that is a combination thereof, but it is not limited thereto. Then, an active metal bronze compound AxMyOz is grafted on the surface of the polymer material, and an oxide-polymer composite AxMyOz-C is formed after dehydration, condensation, and/or other steps are performed.

In the present embodiment, the metal bronze-based compound is grafted on the hydroxyl group (—OH) of the polymer material. Next, one or more of fluoro-containing material including sulfonated perfluoroalkane compounds (e.g. alkyl sulfonic acid/sulfonate fluorosurfactant), sulfonated fluoropolymers (e.g. perfluorosulfonic acid (PFSA)/polytetrafluoroethylene (PTFE) copolymer), or phosphorylated perfluoroalkane compounds (such as alkyl phosphate ester fluorosurfactant) may be assembled with the oxide-polymer composite AxMyOz-C to form a composite structure AxMyOz-C—F.

In the present embodiments, the composite structure AxMyOz-C—F may be formed by co-solvent controlled self-assembly to control the porosity. Highly volatile substances (such as toluene, xylene, methyl ethyl ketone, acetone, propylene glycol methyl ether, propylene glycol methyl ether acetate, water, methanol, alcohol, isopropyl alcohol, or a combination thereof) serve as a first solvent, lowly volatile substances (such as ethylene glycol, diethylene glycol ether, diethylene glycol butyl ether, triethylene glycol, propylene glycol, glycerol, isophorone, N-methylpyrrolidone, dimethyl sulfoxide (DMSO), or a combination thereof) serve as a second solvent, and the first solvent and the second solvent are combined as a composite solvent system. The aforementioned composite AxMyOz-C—F is coated or deposited on a substrate via the composite solvent system. The assembly is performed under a well-controlled environment by using the composite solvent polar system, wherein the ratio of the lowly volatile substances to the highly volatile substances is 5:100. The regarding assembly process may be referred to FIG. 1A to 1E, and it will not be repeated again. A porous structure may be obtained by adjusting the first phase highly volatile assembly, the second phase lowly volatile assembly, and the third phase annealing treatment. For example, the annealing treatment is performed in the atmosphere or in a nitrogen atmosphere at a temperature from 25° C. to 300° C. for 5 minutes to 12 hours. As such, a porous structure may be formed. The average diameter of the holes may be 12.82 μm, and the porosity thereof may be 17.93%. Please refer to FIG. 3, which is an enlarged view illustrating the porous structure in accordance with the present embodiment (Embodiment 5) of the present disclosure.

Embodiment 6: Fabrication of AxMyOz-C—F Composite Structure Membrane with High Degree of Porosity in Microns

The aforementioned polymer material is prepared as a solution with a concentration of 0.001%-20%. For example, the solution may be a 0.1%-15% polyvinyl alcohol solution, a 0.1%-15% methyl cellulose solution, a 0.1%-15% sodium carboxymethyl cellulose solution, a 0.1%-15% hydroxyethyl cellulose solution, a 0.1%-15% hydroxyethyl methyl cellulose solution, a 0.1%-15% hydroxypropyl cellulose solution, a 0.1%-15% hydroxypropyl methylcellulose solution, a 0.1%-15% nanocellulose solution, or a 0.1%-15% aqueous solution that is a combination thereof, but it is not limited thereto. Then, an active metal bronze compound AxMyOz is grafted on the surface of the polymer material, and an oxide-polymer composite AxMyOz-C is formed after dehydration, condensation, and/or other steps are performed.

In the present embodiment, the metal bronze-based compound is grafted on the hydroxyl group (—OH) of the polymer material. Next, one or more of fluoro-containing material including sulfonated perfluoroalkane compounds (e.g. alkyl sulfonic acid/sulfonate fluorosurfactant), sulfonated fluoropolymers (e.g. perfluorosulfonic acid (PFSA)/polytetrafluoroethylene (PTFE) copolymer), or phosphorylated perfluoroalkane compounds (such as alkyl phosphate ester fluorosurfactant) may be assembled with the oxide-polymer composite AxMyOz-C to form a composite structure AxMyOz-C—F.

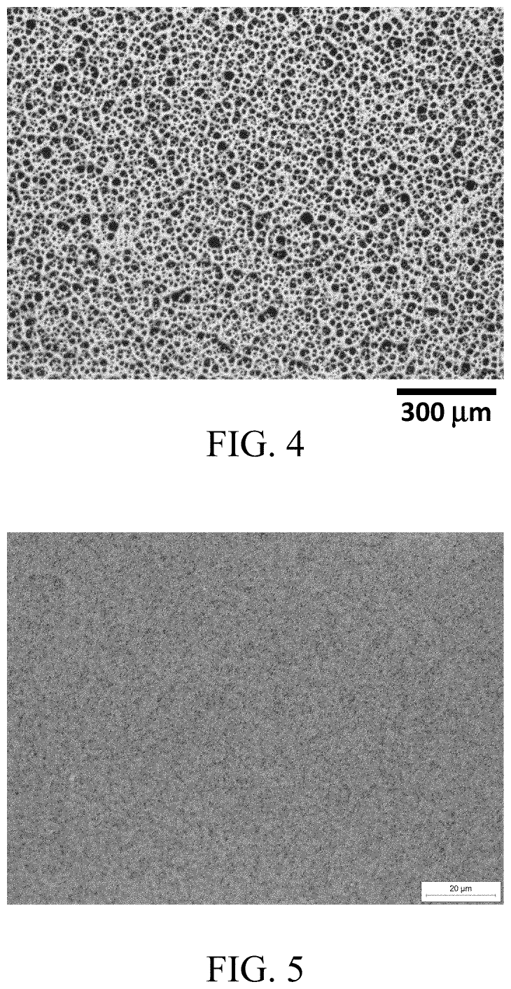

In the present embodiments, the composite structure AxMyOz-C—F may be formed by co-solvent controlled self-assembly to control the porosity. Highly volatile substances (such as toluene, xylene, methyl ethyl ketone, acetone, propylene glycol methyl ether, propylene glycol methyl ether acetate, water, methanol, alcohol, isopropyl alcohol, or a combination thereof) serve as a first solvent, lowly volatile substances (such as ethylene glycol, diethylene glycol ether, diethylene glycol butyl ether, triethylene glycol, propylene glycol, glycerol, isophorone, N-methylpyrrolidone, dimethyl sulfoxide (DMSO), or a combination thereof) serve as a second solvent, and the first solvent and the second solvent are combined as a composite solvent system. The aforementioned composite AxMyOz-C—F is coated or deposited on a substrate via the composite solvent system. The assembly is performed under a well-controlled environment by using the composite solvent polar system, wherein the ratio of the lowly volatile substances to the highly volatile substances is 10:100. The regarding assembly process may be referred to FIG. 1A to 1E, and it will not be repeated again. A porous structure may be obtained by adjusting the first phase highly volatile assembly, the second phase lowly volatile assembly, and the third phase annealing treatment. For example, the annealing treatment is performed in the atmosphere or in a nitrogen atmosphere at a temperature from 25° C. to 300° C. for 5 minutes to 12 hours. As such, a porous structure may be formed. The average diameter of the holes may be 14.34 μm, and the porosity thereof may be 41.66%. Please refer to FIG. 4, which is an enlarged view illustrating the porous structure in accordance with the present embodiment (Embodiment 6) of the present disclosure.

### COMPARATIVE EXAMPLE 3

### Fabrication of Membrane without Composite Solvent System

The aforementioned polymer material is prepared as a solution with a concentration of 0.001%-20%. For example, the solution may be a 0.1%-15% polyvinyl alcohol solution, a 0.1%-15% methyl cellulose solution, a 0.1%-15% sodium carboxymethyl cellulose solution, a 0.1%-15% hydroxyethyl cellulose solution, a 0.1%-15% hydroxyethyl methyl cellulose solution, a 0.1%-15% hydroxypropyl cellulose solution, a 0.1%-15% hydroxypropyl methylcellulose solution, a 0.1%-15% nanocellulose solution, or a 0.1%-15% aqueous solution that is a combination thereof, but it is not limited thereto. Then, an active metal bronze compound AxMyOz is grafted on the surface of the polymer material, and an oxide-polymer composite AxMyOz-C is formed after dehydration, condensation, and/or other steps are performed.

In the present comparative example, the metal bronze-based compound is grafted on the hydroxyl group (—OH) of the polymer material. Next, one or more of fluoro-containing material including sulfonated perfluoroalkane compounds (e.g. alkyl sulfonic acid/sulfonate fluorosurfactant), sulfonated fluoropolymers (e.g. perfluorosulfonic acid (PFSA)/polytetrafluoroethylene (PTFE) copolymer), or phosphorylated perfluoroalkane compounds (such as alkyl phosphate ester fluorosurfactant) may be assembled with the oxide-polymer composite AxMyOz-C to form a composite structure AxMyOz-C—F.

In the present comparative example, assembly of the composite structure AxMyOz-C—F is adjusted by using a highly volatile solvent system. Highly volatile substances (such as toluene, xylene, methyl ethyl ketone, acetone, propylene glycol methyl ether, propylene glycol methyl ether acetate, water, methanol, alcohol, isopropyl alcohol, or a combination thereof) serve as highly volatile solvents. The composite structure AxMyOz-C—F is coated or deposited on a substrate via this highly volatile solvent system, wherein the ratio of the lowly volatile substances to the highly volatile substances is 0:100. The solvent is evaporated under a well-controlled environment, and then a second phase annealing treatment is performed to obtain a dark brown membrane. The annealing treatment is, for example, performed in the atmosphere or in a nitrogen atmosphere at a temperature from 25° C. to 300° C. for 5 minutes to 12 hours. As such, the membrane is formed without a regularly-arranged or obvious porous structure. Please refer to FIG. 5, which is an enlarged view illustrating the membrane in accordance with the present comparative example (Comparative example 3) of the present disclosure.

TABLE 2
The comparison of Embodiments 4-6 and Comparative example 3 for
the fabrication and the description of the holes in microns

| | Embodi- | Embodi- | Embodi- | Comparative |
|---|---|---|---|---|
| | ment 4 | ment 5 | ment 6 | example 3 |
| The ratio of the lowly | 2:100 | 5:100 | 10:100 | 0:100 |
| volatile solvent to | | | | |
| the highly volatile | | | | |
| solvent | | | | |
| Is a porous membrane | Yes | Yes | Yes | No |
| in microns formed? | | | | |
| Degree of porosity | Low | Medium | High | — |
| Average diameter of | 11.94 | 12.82 | 14.34 | — |
| the holes (μm) | | | | |
| Porosity (%) | 8.96 | 17.93 | 41.66 | — |

As set forth above, some embodiments of the present disclosure provide an organic-inorganic composite material with polymers, oxides and fluoro-containing macromolecules. The organic-inorganic composite material may be formed with a plurality of micro holes. The membrane formed by the composite material has both air permeability and dustproof effect, and can effectively protect the sensor from the environment while maintaining the performance of the sensor.

Testing Example 1: Measuring the Capacitance Under Different Relative Humidities

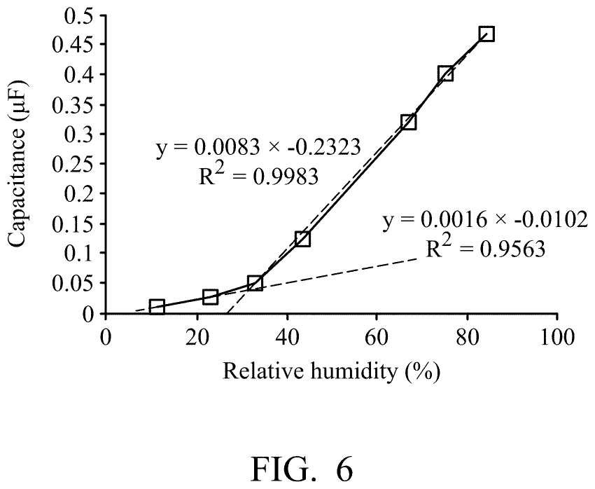

FIG. 6 is a diagram illustrating the relationship between relative humidity and capacitance of the porous structure in accordance with Embodiment 6 of the present disclosure. As shown in FIG. 6, the capacitance of the porous structure may be detected under different relative humidities, and a linear section may be measured when the relative humidity is between 11% and 33%. In addition, another linear section may be measured when the relative humidity is between 33% and 85%.

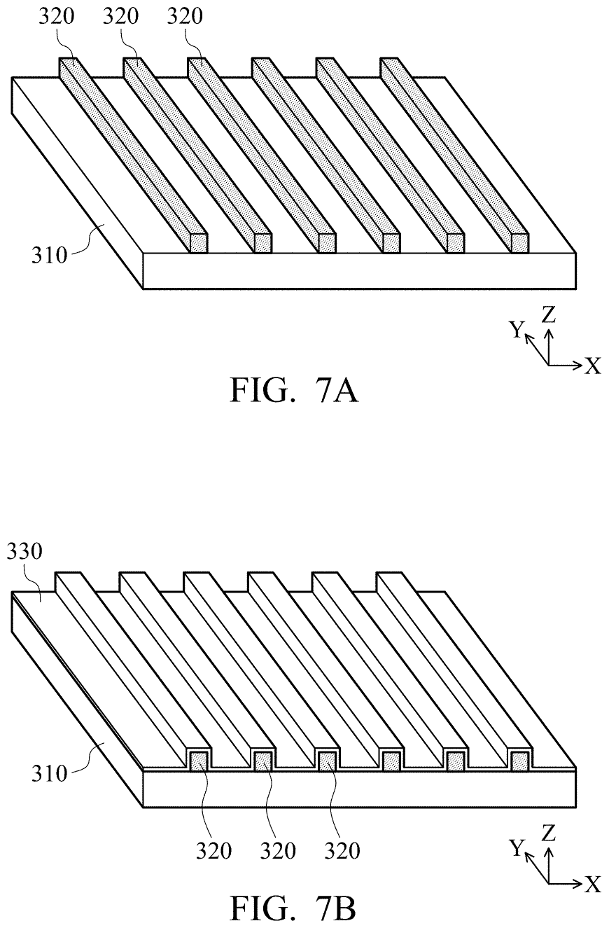

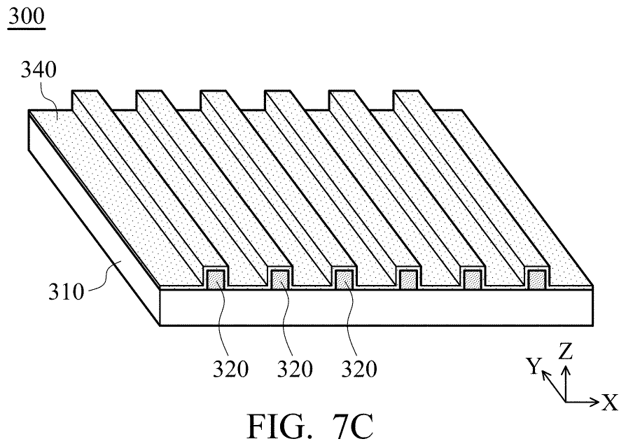

FIG. 7A to 7C are perspective views illustrating a manufacturing process of a gas sensor 300 in accordance with some embodiments of the present disclosure. As shown in FIG. 7A, a substrate 310 is provided, and a plurality of electrodes 320 are disposed on the substrate 310. In the present embodiment, the electrodes 320 extend along the Y direction and are arranged along the X direction in a manner that they are spaced from each other. The electrodes 320 may be configured to detect gases in the environment and obtain parameters such as environment humidity. Then, as shown in FIG. 7B, a membrane 330 is conformally formed on the substrate 310 and the electrodes 320, wherein the membrane 330 includes the above or any other composite structure.

As shown in FIG. 7C, the above or any other annealing process may be performed to form a barrier layer 340. In the present embodiment, the barrier layer 340 may include any of the aforementioned porous structure, but the present disclosure is not limited thereto. By forming a barrier layer with a porous structure on the electrodes 320, the electrodes 320 may be protected without affecting the operation of the electrodes 320. In addition, in the present embodiment, the barrier layer 340 may extend into the space between two adjacent electrodes 320, but it should be appreciated that the barrier layer 340 may not extend into the space between two adjacent electrodes 320 in other embodiments.

While the embodiments and the advantages of the present disclosure have been described above, it should be understood that those skilled in the art may make various changes, substitutions, and alterations to the present disclosure without departing from the spirit and scope of the present disclosure. It should be noted that different embodiments in the present disclosure may be arbitrarily combined as other embodiments as long as the combination conforms to the spirit of the present disclosure. In addition, the scope of the present disclosure is not limited to the processes, machines, manufacture, composition, devices, methods and steps in the specific embodiments described in the specification. Those skilled in the art may understand existing or developing processes, machines, manufacture, compositions, devices, methods and steps from some embodiments of the present disclosure. Therefore, the scope of the present disclosure includes the aforementioned processes, machines, manufacture, composition, devices, methods, and steps. Furthermore, each of the appended claims constructs an individual embodiment, and the scope of the present disclosure also includes every combination of the appended claims and embodiments.

What is claimed is:

1. A barrier layer, comprising:
a porous structure, comprising:
a polymer material;
an oxide, wherein a chemical bond is formed between the polymer material and the oxide; and
a fluoro-containing material, wherein the fluoro-containing material, the polymer material and the oxide are assembled as a composite structure.

2. The barrier layer as claimed in claim 1, wherein the polymer material comprises repeating units as follows: 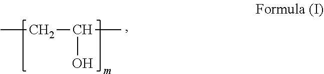
wherein m is an integer in a range from 1 to 10000.

3. The barrier layer as claimed in claim 1, wherein the polymer material comprises repeating units as follows: 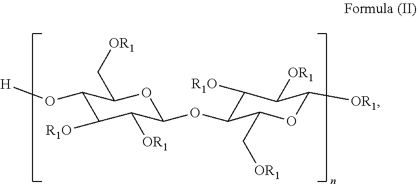
wherein m is an integer in a range from 1 to 10000, R₁ is (CH₂)ᵢH, (OC₂H₄)ⱼH, (OC₃H₆)ₖH, or a combination thereof, i is an integer in a range from 0 to 24, j is an integer in a range from 0 to 18, and k is an integer in a range from 0 to 12.

4. The barrier layer as claimed in claim 3, wherein the plurality of R₁ are the same as or different from each other, or partially the same but partially different.

5. The barrier layer as claimed in claim 1, wherein the oxide comprises a unit as follows:
AxMyOz Formula (III),
Wherein A comprises at least one cation, M comprises at least one of transition metal ions, metalloid ions and carbon ions, and values of x, y and z equalize charge number of the Formula (III).

6. The barrier layer as claimed in claim 5, wherein A comprises at least one of hydrogen ion, alkali metal ion, alkaline earth metal ion, rare earth metal ion, and ammonium ion.

7. The barrier layer as claimed in claim 5, wherein M comprises at least one of tin, titanium, zirconium, cerium, hafnium, molybdenum, tungsten, vanadium, copper, iron, cobalt, nickel, manganese, niobium, tantalum, rhenium, ruthenium, platinum, silicon, boron, germanium, arsenic, and carbon.

8. The barrier layer as claimed in claim 1, wherein the fluoro-containing material comprises at least one of perfluoroalkyl, polytetrafluoroethylene, sulfonic acid, and phosphoric acid formed by fluorocarbons.

9. The barrier layer as claimed in claim 1, wherein the oxide is grafted on a hydroxyl group of the polymer material.

10. A gas sensor, comprising:
a substrate;
a plurality of electrodes formed on the substrate; and
the barrier layer as claimed in claim 1 formed on the electrodes.

11. The gas sensor as claimed in claim 10, wherein the barrier layer extends into the space between two of the electrodes.

12. A method for forming a barrier layer, comprising:
providing a composite solvent system, wherein the composite solvent system comprises a first solvent and a second solvent, and the volatility of the first solvent is different from the volatility of the second solvent;
adding a composite into the composite solvent system;
coating the composite solvent system on a substrate after adding the composite; and
forming a porous structure on the substrate after the first solvent and the second solvent are volatilized.

13. The method as claimed in claim 12, wherein the first solvent is volatilized faster than the second solvent.

14. The method as claimed in claim 13, wherein the amount of the first solvent is greater than the amount of the second solvent.

15. The method as claimed in claim 14, wherein the ratio of the second solvent to the first solvent is from 2:100 to 10:100.

16. The method as claimed in claim 12, wherein the first solvent comprises at least one of toluene, xylene, methyl ethyl ketone, acetone, propylene glycol methyl ether, propylene glycol methyl ether acetate, water, methanol, alcohol, and isopropyl alcohol.

17. The method as claimed in claim 12, wherein the second solvent comprises at least one of ethylene glycol, diethylene glycol ether, diethylene glycol butyl ether, triethylene glycol, propylene glycol, glycerol, isophorone, N-methylpyrrolidone, and dimethyl sulfoxide.

18. The method as claimed in claim 12, further comprising:
performing an annealing treatment to form the porous structure, wherein the annealing treatment is performed in the atmosphere or in a nitrogen atmosphere at a temperature from 25° C. to 300° C. for 5 minutes to 12 hours.

19. The method as claimed in claim 12, wherein the porous structure is conformally formed on the substrate.

20. The method as claimed in claim 12, wherein the composite is formed by assembling a polymer material, an oxide, and a fluoro-containing material.
